(19) United States
(12) Patent Application Publication
(10) Pub. No.: US 2021/0388384 A1
(43) Pub. Date: Dec. 16, 2021

(54) VECTORS

(71) Applicant: AUTOLUS LIMITED, London (GB)

(72) Inventors: Martin Madill, London (GB); Richard Beswick, London (GB); Ekaterini Kotsopoulou, London (GB)

(21) Appl. No.: 17/283,833

(22) PCT Filed: Oct. 11, 2019
(86) PCT No.: PCT/GB2019/052905

§ 371 Date: Apr. 8, 2021

(30) Foreign Application Priority Data
Oct. 12, 2018 (GB) 1816644.7

(51) Int. Cl. C12N 15/86; C12N 5/0783; A61K 35/17; C07K 14/005

(52) U.S. Cl. CPC C12N 15/86; C12N 5/0636; A61K 35/17; C12N 2740/16222; C12N 2740/13043; C12N 2740/16043; C12N 2510/00; C07K 14/005

(57) ABSTRACT

The present invention provides a kit of vectors comprising a first and second viral vector, wherein the first viral vector comprises a transgene of interest (TOI), and presence of the second viral vector in a host cell is required for integration of the first viral vector TOI into the host cell genome.

### FIELD OF THE INVENTION

The present invention relates to a kit of vectors. For example, retroviral vectors for transducing a cell. In particular, the invention relates to a kit comprising a first and second viral vector, wherein the first vector comprises a transgene of interest (TOI), and presence of the second vector in a host cell is required for integration of the first vector TOI into the host cell genome.

The invention also relates to pharmaceutical compositions and methods for treating/preventing a disease comprising administering compositions of such cells, and methods for making a kit of vectors.

### BACKGROUND TO THE INVENTION

Viral vectors have been used to transduce primary cells, such as T-cells or haematopoietic stem cells (HSCs), to express polypeptides of interest for decades. These vectors exploit the specialised molecular mechanisms evolved in viruses to efficiently transfer their genome inside the cell they infect. However, they have a finite transfer capacity. For retroviral vectors this is generally considered to be around 8 to 10 kilobases (kb). The limit is due to the packaging efficiency being inversely proportional to the insert size.

Other potential non-viral mechanisms of cell-based gene therapy with a higher insert capacity are known, but these are often hampered by low efficiency of transduction and/or toxic effects that yield low T cell numbers.

To transduce a large insert size into a cell whilst maintaining high efficiency, the genes encoded on a viral vector may be split into two or more separate vectors. Each vector is made separately and all vectors are then used to transduce the cells. However, this multiple transduction approach often results in cells that are transduced with some but not all of the desired vectors. This leads to a non-uniform cell population comprising different vector integrants, which will not express all of the desired genes.

It is desirable to only use the transduced cells that contain the desired genes, and hence with current methods, known cell selection/cell sorting steps are required to obtain a homogeneously transduced population.

A commonly used cell sorting/selection method is by flow cytometry, in particular, fluorescent activated cell sorting (FACs) where researchers label specific cells within a mixture using fluorescently tagged antibodies that bind selected cell-surface molecules. However, intrinsic cell fluorescence has limited researchers' ability to distinguish positive and negative signals and many researchers have expressed concerns about the cell throughput of typical FACS systems. An increasing number of researchers have started to apply immunomagnetic cell sorting (MACS) methods to isolate specific cells from a mixture. Like FACS, MACS relies on antibodies to label cells but the antibodies are attached to biodegradable super para magnetic beads that range in diameter from several micrometers to tens of nanometers. Thus, rather than rely on a laser to identify and an electrical charge to separate appropriately labeled cells, scientists use a powerful magnet to hold cells in the reaction chamber while washing away unlabeled cells. However, MACS is also not without its problems as using paramagnetic beads limits researchers' ability to perform multiplex reactions, unlike FACS, where they can use different fluorophores. In many cases, researchers use a combination of MACS and FACS to achieve their goals.

Both methods require a relatively costly and complex series of steps and specialised equipment, which ultimately delays the manufacturing process.

There is a need to provide improved methods of transducing primary cells with large insert sizes by generating homogeneously transduced cell populations without the need of time-consuming, expensive and cell-yield jeopardising cell selection steps.

### DESCRIPTION OF THE FIGURES

FIG. 1: Viral vector virion structure. A: Surface envelope protein (SU); B: Transmembrane envelope protein (TM); C: Membrane (M); D: Matrix protein (MA); E: Capsid (CA); F: RNA genome, bound by nucleocapsid (NC); G: Integrase (IN); H: Reverse Transcriptase (RT); I: Protease (PR).

FIG. 2: Co-transduction of GFP positive cells. Schematic of transduction with an integration-deficient vector (first vector) and an integration-competent vector (second vector). The integration-deficient viral vector may be produced by a mutant Gag pol e.g. deletion of IN. The integration-competent viral vector may be produced by wild type (WT) Gag pol. The IN present in the integration-competent viral vector permits successful integration of GFP RNA in the integration-deficient viral vector, as well as the APRIL-CAR RNA into the host cell genome.

FIG. 3: Co-transduction of positive cells. Schematic of transduction with an integration-deficient vector (first vector) and a reverse transcription-deficient vector (second vector). The integration-deficient vector is produced by a mutant Gag pol e.g. deletion of IN. The reverse transcription-deficient vector is produced by a different mutant Gag pol e.g. deletion of RT. The combination of both vectors having deficiencies in integration and reverse transcription activities, respectively, permits successful of GFP RNA in the integration-deficient viral vector, as well as the APRIL-CAR RNA into the host cell genome.

FIG. 4: Gag pol constructs of a viral vector (MLV).

A-B: Wild type (WT) Gag pol. B displaying WT Gag pol viral proteins MA; RNA binding phosphoprotein (RP); CA; NC; PR, RT and IN. C-K: Mutant Gag pol constructs which produce either an integration-deficient viral vector and/or a reverse transcription-deficient viral vector. C: Mutant Gag Pol lacking IN. D: Mutant Gag pol lacking RT. E: Mutant Gag pol lacking both IN and RT. F: Mutant Gag pol comprising RP amino acid substitution S192A, abolishing RT activity. G: Mutant Gag pol comprising RP amino acid substitution S192D, abolishing RT activity. H: Mutant Gag pol comprising IN amino acid substitutions D184N and/or K376A, abolishing IN activity. I: Mutant Gag pol comprising RP amino acid substitution S192A or S192D, and IN amino acid substitution(s) D184N and/or K376A. J: Mutant Gag pol comprising RP amino acid mutation of S192A or S192D, and deletion of IN. K: Mutant Gag pol comprising IN amino acid substitution(s) D184N and/or K376A and deletion of RT. Both RT activity and IN activity is abolished in mutant Gag pol constructs: E, I, J and K.

FIG. 5: Gag pol constructs of a viral vector (MLV).

A-B: WT Gag pol, B displaying WT viral proteins MA; RP; CA; NC; PR, RT and IN. C: Mutant Gag pol comprising an additional IN in the pol region of Gag pol. D: Mutant Gag pol comprising an additional IN in the Gag region of the Gag pol polyprotein. E: Mutant Gag pol comprising an additional RT in the pol region of the Gag pol. F: Mutant Gag pol comprising additional RT in the Gag region of the Gag pol.

FIG. 6: Vector combination table showing TOI expression by cells resulting from single or double transduction of vectors of a kit, where the first and second vectors comprise a first and second TOI respectively.

FIG. 7: Bar graph and table showing percentage of GFP positive HEK293T cells, (from an integration-deficient vector). Bar graph of GFP positive cells (%) transduced with an integration-deficient (IN−) viral vector alone (first viral vector) comprising a gene encoding GFP; cells transduced with an integration-competent viral vector alone (second viral vector) comprising wild type Gag pol and a gene encoding RQR8, and cells transduced with both of the viral vectors.

The table shows that only when cells are transduced with both the first and second viral vectors is a significant yield of the IN−deficient TOI (GFP) expression achieved.

FIG. 8: Bar graph and table showing percentage of RQR8 positive HEK293T cells (from an integration-competent vector). Bar graph of RQR8 positive cells (%) transduced with an integration-deficient (IN−) viral vector alone (first viral vector) comprising a gene encoding GFP, cells transduced with an integration-competent viral vector (second viral vector) comprising wild type Gag pol and a gene encoding RQR8), and cells transduced with both first and second viral vectors.

The table shows that significant RQR8 expression is achieved when the cells are transduced with the second viral vector or both the first and second viral vectors. Significant levels of RQR8 expression are not achieved when cells are transduced with the first viral vector alone.

FIG. 9: Histogram of GFP expression (from a integration-deficient vector encoding GFP). (A) HEK293T Cells transduced with an integration-deficient viral vector (IN−); (B) HEK293T cells transduced with an integration-competent viral vector (IN+); or (C) HEK293T cells transduced with both first and second viral vectors. Only when the cells are transduced with both the first and second viral vectors are they shown to be GFP positive (28%).

FIG. 10: Bar graph and table showing percentage of GFP positive T cells (from an integration-deficient vector).

Bar graph of GFP positive T cells (%) transduced with an integration-deficient (IN−) viral vector alone (first viral vector) comprising a gene encoding GFP, an integration-competent viral vector alone (second viral vector) comprising wild type Gag pol and a gene enconding RQR8, and cells transduced with both the first and second viral vectors.

The table shows that only when the T cells are transduced with both the first and second viral vectors is a significantly higher yield of GFP expression achieved, compared with the yield achieved with either the first or second viral vectors alone. This is because the second viral vector is an integration competent vector (WT Gag pol) is able to rescue the first vector's integration deficiency.

FIG. 11: Bar graph and table showing percentage of RQR8 positive T cells (from an integration-competent vector). Bar graph of RQR8 positive T cells (%) transduced with the first viral vector (integration-deficient IN−) comprising a gene encoding GFP, T cells transduced with the second viral vector (integration-competent) comprising wild type Gag pol and a gene encoding RQR8, and T cells transduced with both the first and second viral vectors.

The table shows that significant RQR8 expression is achieved when the cells are transduced with the first and second viral vectors or only the second vector. In contrast, the level of RQR8 expression is not significant when cells are transduced with the first vector alone.

FIG. 12: Bar graph and table showing percentage of CD19-CAR positive T cells (from an integration-deficient vector).

Bar graph of CD19-CAR positive T cells (%) transduced with an integration-deficient (IN−) viral vector alone (first viral vector) comprising a gene encoding CD19-CAR, an integration-competent viral vector alone (second viral vector) comprising wild type Gag pol and a gene encoding RQR8, and cells transduced with both the first and second viral vectors.

The table shows that only when the T cells are transduced with both the first and second viral vectors is a significantly higher yield of CD19-CAR expression achieved, compared with the yield achieved with either the first or second viral vectors alone. This is because the second viral vector, an integration competent vector (WT Gag pol), is able to rescue the first vector's integration deficiency.

FIG. 13: Bar graph and table showing percentage of RQR8 positive T cells (from an integration-competent vector). Bar graph of RQR8 positive T cells (%) transduced with the first viral vector (integration-deficient IN−) comprising a gene encoding CD19-CAR, T cells transduced with the second viral vector (integration-competent) comprising wild type Gag pol and a gene encoding RQR8, and T cells transduced with both the first and second viral vectors.

The table shows that significant RQR8 expression is achieved when the cells are transduced with the first and second viral vectors or only the second vector. In contrast, the level of RQR8 expression is not significant when cells are transduced with the first vector alone.

### SUMMARY OF INVENTION

The inventors have developed a kit of vectors, comprising first and second vectors, at least one of which comprises a transgene of interest (TOI). In order for the TOI to integrate into a host cell genome, the cell has to be successfully transduced with both vectors. This is because the vector comprising the TOI is an integration and/or reverse transcription-deficient viral vector, whereas the other vector is an integration and/or reverse transcription-competent viral vector.

An integration-competent vector in the presence of an integration-deficient vector comprising a TOI restores the integration activity of the deficient vector, thus permitting integration of the TOI into the host cell to be stably expressed. Similarly, a reverse transcription-competent vector in the presence of a reverse transcription deficient vector comprising a TOI, restores the reverse transcription activity of the deficient vector, permitting integration of the TOI into the host cell to be stably expressed.

Thus, in a first aspect, the present invention provides a kit of vectors comprising a first vector and a second vector
wherein the first viral vector comprises a first transgene of interest (TOI), and
presence of the second viral vector in a host cell is required for integration and expression of the first viral vector TOI into the host cell genome.

When the first viral vector is integration-deficient, the second viral vector is integration-competent.

When the first viral vector of the kit is integration-deficient, the first viral vector may lack an integrase (IN).

When the first viral vector of the kit is integration-deficient, the first viral vector may comprise a truncated IN.

When the first viral vector of the kit is integration-deficient, the first viral vector may comprise an IN with one or more amino acid substitution(s).

When IN derives from a Moloney murine leukemia virus (MLV) viral vector, the amino acid substitutions may be selected from D184N and/or K376A.

When IN derives from a Human Immunodeficiency virus (HIV) viral vector, the amino acid substitution may be D116A.

Additionally or alternatively, the first viral vector may be reverse transcription-deficient and the second viral vector may be reverse transcription-competent.

When the first viral vector of the kit is reverse transcription-deficient, the first viral vector may lack reverse transcriptase (RT).

When the first viral vector of the kit is reverse transcription-deficient, the first viral vector may comprise a truncated RT.

When the first viral vector of the kit is reverse-transcription deficient, the first viral vector may comprise a RNA Binding Phosphoprotein (RP) with one or more amino acid substitutions. The one or more amino acid substitution abolishes RT activity. When the RP derives from MLV, the amino acid substitution may be selected from S192D or S192A.

In a first embodiment of the first aspect of the invention, the first viral vector is integration and/or reverse transcription-deficient, and the second viral vector is both integration and reverse-transcription competent.

The second viral vector may comprise a second TOI. When cells are transduced with the kit of vectors, they will either express both the first and second TOIs (when the cell is successfully transduced with both vectors), or the second TOI only (when the cell is transduced with the second vector alone). If a cell is transduced with the first vector alone it will not express either transgene.

In a second embodiment of the first aspect of the invention, there is provided a kit of vectors comprising a first vector and a second vector in which the presence of both vectors in a cell following transduction is necessary for integration of a transgene of interest. For example, when the first vector is integration-deficient, the second vector may be reverse transcription-deficient and integration-competent.

Alternatively, when the first vector is reverse transcription-deficient, the second vector may be integration-deficient and reverse transcription-competent. In this embodiment, when the second viral vector comprises a second TOI and cells are transduced with the kit of vectors, the cells will either express both the first and second TOIs (when the cell is successfully transduced with both vectors), or no transgenes (when the cell is transduced with either the first vector or the second vector alone). If a cell is transduced with the either vector alone it will not express either transgene because it will lack either a functional integrase or reverse transcriptase.

The TOI may encode a Chimeric Antigen Receptor (CAR) and/or an enhancer. Examples of enhancers include but are not limited to cytokines or transcription factors. In the first embodiment of the first aspect of the invention, the first TOI may encode a CAR or a transgenic TCR and the second TOI may encode an enhancer molecule.

The kit of vectors of the present invention may comprise a first, second and third viral vector.

In a second aspect, the present invention provides a cell transduced with a kit of vectors of the first aspect. The cell may be an immune cell such as a T cell, natural killer (NK) cell or a hematopoietic stem cell (HSC).

In a third aspect, the present invention provides a method for making the cell of the second aspect, which comprises the step of transducing a cell with the kit of vectors of the first aspect.

In a fourth aspect, the present invention provides a method for preparing a composition of cells according to the second aspect, which comprise the following steps
a) transducing a cell-containing sample with a kit of vectors of the first aspect;
b) culturing the cell containing sample to enable integration of all the vectors of the kit
c) preparing a composition of TOI expressing cells.

In a fifth aspect, the present invention provides a composition comprising a plurality of cells according to the second aspect. The composition may be for use in treating and/or preventing a disease.

In a sixth aspect, the present invention provides a method for treating and/or preventing a disease, which comprises the step of administering a pharmaceutical composition of the fifth aspect. The method of the sixth aspect comprises the following steps:
a) isolation of a cell-containing sample from a subject
b) transducing the cell-containing sample with a kit of vectors of the first aspect
c) culturing the cell-containing sample to enable integration of all the vectors of the kit,
d) administering the TOI expressing cell-containing sample to the subject.

In a seventh aspect, the present invention provides the use of a pharmaceutical composition according to the sixth aspect of the invention in the manufacture of a medicament for the treatment and/or prevention of a disease. The disease may be cancer.

In an eighth aspect, the present invention provides a method of making a kit of vectors according to the first aspect, comprising generating the first viral vector by using an integration-deficient and/or reverse transcription-deficient Gagpol.

Provided herein is the method of making a kit of vectors according to the first aspect when the first viral vector is integration-deficient, comprising the following steps:
a) generating the first viral vector by using a sequence encoding an integration-deficient Gagpol; and
b) generating the second viral vector by using a sequence encoding a reverse-transcription deficient Gagpol.

Provided herein is the method for making a kit of vectors according to the first aspect, when the first viral vector is reverse transcription-deficient, comprising the following steps:
a) generating the first viral vector using a sequence encoding an reverse transcription-deficient Gagpol; and
b) generating the second vector using a sequence encoding a integration-deficient Gagpol.

Surprisingly, the inventors have found that an integration-deficient vector (e.g. first vector) is capable of having its integration activity rescued by the IN present in an integration-competent vector (e.g. second vector). Similarly, they found that a reverse transcription-deficient vector (first vector) is capable of having its reverse transcription activity rescued by the RT present in a reverse transcription-competent vector (e.g., second vector) of a kit of vectors.

This rescuing effect enables successful integration of first TOI of the deficient (integration- and/or reverse transcription-deficient) vector intothe host cell genome, which would have otherwise not have been possible.

Where the vectors in the kit each express a transgene, this effect therefore also provides a method of ensuring a cell population expresses a mixture of the first and second TOIs of the co-transduced first and second vectors or the second TOI alone (first embodiment) or only expresses a mixture of the first and second TOI (second embodiment), without the need to sort the cells. Importantly, the method removes the possibility of a cell population expressing only the first TOI because the first vector comprising the first TOI is a deficient vector and can not replicate or integrate the TOI into the host cell without presence of the the second vector.

This is advantageous as it eliminates the need to carry out the time-consuming, complex and expensive cell sorting and selection steps of the resulting cell population and can improve the yield of TOI expressing cells.

This effect is particularly advantageous where the first TOI is an enhancer molecule e.g., such as a cytokine, which may be undesirable or even dangerous to be expressed by the host cell without the second TOI e.g., such as a CAR.

### DETAILED DESCRIPTION

Retrovi Ruses

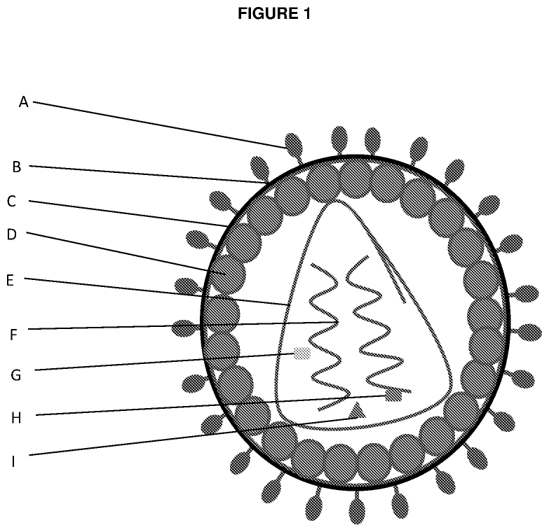

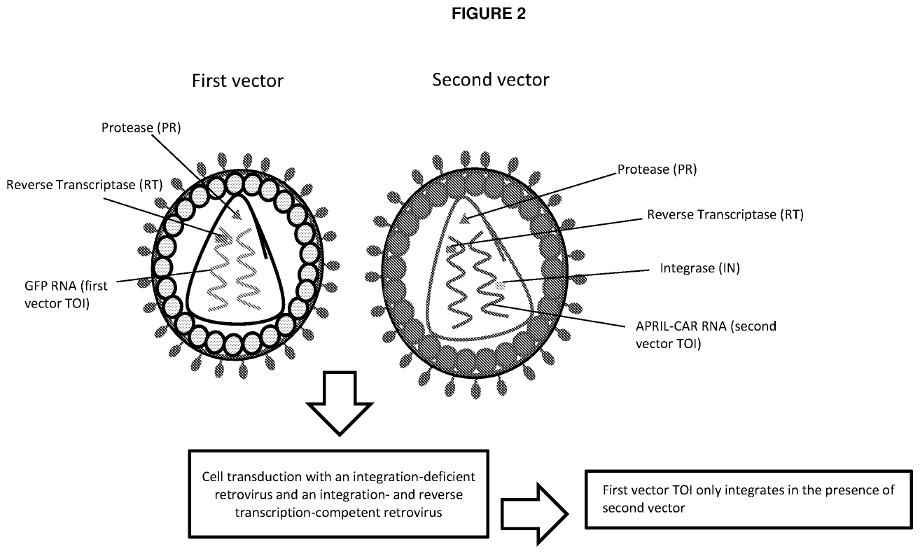

Retroviruses are single stranded RNA enveloped viruses mainly characterized by the ability to “reverse-transcribe” their genome from RNA to DNA. Virions measure 100-120 nm in diameter and contain two copies of a positive strand RNA genome complexed with the nucleocapsid (NC) proteins (FIG. 1). The genome is enclosed in a protein capsid (CA) that also contains enzymatic proteins, namely the reverse transcriptase (RT), the integrase (IN) and proteases (PR), all required for viral infection. Matrix (MA) proteins form a layer outside the capsid core that interacts with the viral envelope, a lipid bilayer derived from the host cellular membrane, which surrounds the viral core particle. Anchored on this bilayer, are the viral envelope glycoproteins responsible for recognizing specific receptors on a host cell and initiating the infection process. These envelope proteins are formed by two subunits: the transmembrane (TM) that anchors the protein into the lipid membrane and the surface (SU) which binds to the cellular receptors.

The retroviral genome contains three primary open reading frames: Gag (group specific antigen), Gag-Pol (group specific antigen—protease—polymerase) by read through of a stop codon, and Env (envelope). These are initially translated into precursor polyproteins, which are subsequently processed by cellular or viral proteases into mature proteins. The Gag precursor is cleaved into the matrix (MA), capsid (CA), nucleocapsid (NC) and p12 proteins. The Pol polyprotein is cleaved down to yield the protease, viral reverse transcriptase (RT) and integrase (IN) and enzymes. The proteases are required for the cleavage of the individual Gag and Gag-Pol proteins during particle assembly, budding and maturation. In contrast, Env is cleaved into the surface and transmembrane proteins by furin within the Golgi apparatus while being trafficked to the cell surface. The RT catalyses the reverse transcription of the viral genome from RNA to DNA following entry into the target cell, and IN is responsible for integrating the proviral DNA into the host cell genome. Without the reverse transcription and integration activity, the proviral DNA is not able to integrate into the host cell genome.

Based on the genome structure, retroviruses are classified into simple retroviruses such as MLV (murine leukemia virus); or complex retroviruses such as HIV or EIAV. In addition to Gag, Pol and Env, complex retroviruses, such as lentiviruses, contain additional gene products that are translated using spliced mRNA and include the regulatory proteins Tat and rev, which promote viral gene expression, and accessory proteins vpr, vpu and nef involved in virulence, assembly of infectious particles and replication.

The retroviral genome also contains cis-acting nucleic acid sequence elements, many of which are clustered near the 5′ untranslated region divided into U3, R and U5. This region is highly structured, containing several hairpin loops, and is absolutely required in cis for the packaging and reverse transcription of the genome. The features found here include the primer binding site (PBS), which is required for the initiation of reverse transcription, and the packaging signal (ψ). This packaging domain also contains the main splice donor (SD) site. The 3′ untranslated region contains the polypurine tract (PPT), which is utilised during the synthesis of plus-strand DNA, as well as copies of the U3 and R regions. Thus, the proviral DNA is flanked by two incomplete Long Terminal Repeats (LTRs) that are restored prior to integration of the viral genome into the host cell chromatin.

During the process of infection, a retrovirus initially attaches to a specific cell surface receptor via the envelope glycoprotein. On entry into the susceptible host cell, the viral particles are partially uncoated in the cytoplasm and begin the reverse transcription of the RNA genomes by the virally delivered RT in the host cell cytoplasm. The resulting partially double-stranded DNA is transported to the host cell nucleus where it subsequently integrates into the chromosomal DNA assisted by the Integrase (IN) enzyme. At this stage, it is typically referred to as the provirus. The provirus is stable in the host chromosome during cell division and is transcribed like other cellular proteins. Transcripts of the viral genome are exported into the cytoplasm and some are spliced to produce all necessary viral proteins. The retroviral genome is packaged into the virions as two copies of single-stranded RNA, and progeny virions form by budding from the cell membrane. In this way, host-cell derived membrane proteins become part of the retroviral particle.

Reverse transcription of the retroviral genome is a key step in the process of infection. It is catalysed by the viral heterodimeric RT enzyme, which has both RNaseH and DNA polymerase functions, using either RNA or DNA as a template and removing ribonucleotides. The process begins by the initiation of the minus-strand DNA synthesis from tRNA annealed to the PBS. DNA synthesis proceeds to the 5′ end of the genome to generate the U5 and R sequences, at which point the RNase function of the RT digests the RNA portion of the DNA-RNA hybrid and the short ssDNA fragment is translocated to the 3′ end of the genome where it hybridises with the short homologous R region. The annealing of the minus-strand DNA creates a suitable primer-template structure for DNA synthesis. The RT continues to elongate the minus-strand DNA until the primer binding site at the 5′ end of the genome, and this is accompanied by degradation of the template RNA. The RNA fragment bound to the PPT in the distal region of the genome is not removed by RNaseH and acts as a main primer for the plus-strand synthesis. RNaseH also displaces the tRNA primer, allowing the plus- and minus-strand DNAs to hybridise, and strand syntheses to go to completion.

The integration of linear retroviral DNA, like reverse transcription, is a crucial and defining feature of the life cycle. The integration of the viral genome is mediated by the pre-integration complex (PIC). The proviral dsDNA is transported into the nucleus as part of the PIC, which additionally comprises IN, capsid and p12 as well as host cell proteins.

The processing and integration of the viral genome is catalysed by the IN, and although the chromosomal location of the integration event is not defined, the steps in the viral genome processing are. IN begins by removing the invariant CA dinucleotide from the blunt ends of the viral genome to create 5′ overhangs. It then creates a staggered cut in the genomic DNA and mediates the strand transfer of the 3′ overhangs to the matching recessed ends in the genomic DNA. Cellular repair machinery assists by filling the gaps and covalently joining the free DNA ends, hence the process requires both viral and host factors to go to completion.

Retroviral Vectors

Retroviruses and lentiviruses may be used as a basis for a vector or a delivery system for the transfer of a transgene of interest (TOI), or a plurality of TOIs, into a target cell. The transfer can occur in vitro, ex vivo or in vivo. Viral vectors of the present invention may comprise a TOI(s), which may encode any transgene desired to be expressed in the cell. For example, a T cell receptor or a chimeric antigen receptor or a suicide gene.

Retroviruses can be converted into retroviral vectors by splitting the different parts of the genome onto separate plasmids, resulting in replication-deficient vector particles capable of only one round of cell entry. This is achieved by providing the different components required for vector assembly in trans; the Env gene on one plasmid, the Gag-Pol coding region on another plasmid, and the TOI(s) on a third plasmid. Lentiviral vector production requires a fourth plasmid containing the Rev accessory genes. Only the TOI is accompanied in cis by the LTRs, PBS and packaging signal, and as such is packaged into the recombinant vector particles together with the RT and IN enzymes. The vectors carry the RT and IN enzymes required for transduction only as proteins and not as genetic material, resulting in vectors capable of reverse transcribing and integrating their genome containing the TOI into the target cells but not capable of generating infectious progeny.

Gamma-retroviral vectors, commonly designated retroviral vectors, were the first viral vector employed in gene therapy clinical trials in 1990 and are still one of the most used. More recently, the interest in a sub-family of retroviral vectors, lentiviral vectors, derived from complex retroviruses such as the human immunodeficiency virus (HIV), has grown due to their ability to transduce non-dividing cells. The most attractive features of retroviral and lentiviral vectors as gene transfer tools include the capacity for large genetic payload (up to 9 kb), minimal patient immune response, high transducing efficiency in vivo and in vitro, and the ability to permanently modify the genetic content of the target cell, sustaining a long-term expression of the delivered gene. While both lentiviruses and gamma-retroviruses may use the same gene products for packaging (i.e., Gag, Pol, and Env), the isoforms of these proteins differ so they are not interchangeable. General envelope plasmids, such as VSV-G, however, may be used across both systems.

The retroviral vector may be based on any suitable retrovirus which is able to deliver genetic information to eukaryotic cells. For example, the retroviral vector may be an alpharetroviral vector, a gammaretroviral vector, a lentiviral vector or a spumaretroviral vector. Such vectors have been used extensively in gene therapy treatments and other gene delivery applications. Retroviral vectors are commonly produced by transfection of a host cell line such as Human Embryonic Kidney 293 (HEK293), using a three-plasmid system. The plasmids may be transiently transfected into the host cell line, resulting in a vector production method called transient transfection. The plasmids may also be stably integrated into the genome of the host cell line, resulting in a vector production method called stable producer cells. When only the Gag-pol and Env plasmids are stably integrated into the host cell genome, the production method utilises a packaging cell line. The genome plasmid can be transiently transfected into the packaging cell line resulting vector production. When all three plasmid required for vector production are stably integrated into the host cell genome, the production method utilises a producer cell line and the vector can be harvested from the supernatant without the need for further transfection.

The most attractive features of retroviral and lentiviral vectors as gene transfer tools include the capacity for large genetic payload (up to about 8-10 kb), minimal patient immune response, high transducing efficiency in vivo and in vitro, and the ability to permanently modify the genetic content of the target cell, sustaining a long-term expression of the delivered gene. To further increase/expand the amount of genetic information that can be integrated into the host cell, multiple vectors, such as a kit of vectors, can be co-transduced into cells.

Kit of Vectors

The first aspect of the invention provides a kit of vectors. The kit of vectors comprises more than one vector. The kit of vectors comprises at least a first vector and a second vector. The kit may contain two, three, four, five or more vectors. The number of vectors in the kit of is related to the total size of the insert desired to be transduced into the host cell: where the total insert size is large, it may be split into a larger number of vectors.

Splitting the complete insert between multiple vectors offers advantages over a vector comprising multiple TOIs of interest within the same cassette. The single vector arrangement can result in problems with efficiency of translation and transcription due to, for example, promoter interference whereby one promoter dominates and causes silencing of the second promoter.

The vectors in the kit deliver at least one desired transgene of interest (TOI) to the host cell such that, when a cell is transduced with the vectors of the kit of the present invention, the desired TOI is expressed by the cell. The combination of vectors of the present invention eliminates the need to perform a cell sorting/selective step that is usually required.

At least one of the vectors of the kit comprise at least one TOI. The first vector comprises a first TOI. Each of the first and second vectors of the kit may comprise a first and second TOI, respectively. Each vector may comprise at least one TOI. Alternatively, each vector of the kit may comprise at least two TOIs.

Two co-expressed TOIs may be advantageous, for example, wherein one may enhance expression of the other. For example, a cytokine may enhance the expression of a Chimeric Antigen Receptor (CAR). Expression of first TOI on the first vector (deficient) will depend on the presence of the second vector (competent) which comprises the second TOI. Absence of the second vector will not rescue the deficiency of the first vector and therefore the cell will not express the first TOI (e.g. cytokine). Blocking expression of the first TOI may advantageous where expression of the first TOI e.g. cytokine without the second TOI e.g. a CAR is undesirable e.g. may contribute to risk of cytokine storm.

In a first embodiment of the first aspect of the invention, the first viral vector is integration and/or reverse transcription-deficient, and the second viral vector is both integration and reverse-transcription competent.

When both the first and second vector comprise a transgene and the kit of vectors is used to transduce cells, the resultant cell population will comprise cells expressing both transgenes (resulting from successful transduction with both transgenes); cells expressing just the second transgene (resulting for transduction with the second vector only); and cells expressing no transgenes (no transduction of either vector).

In this embodiment, the combination of vectors ensures that there are no cells in a transduced cell population which express the first transgene alone.

In a second embodiment of the first aspect of the invention, either:
the first vector is integration-deficient and reverse transcription-competent and the second vector is reverse transcription-deficient and integration-competent; or
the first vector is reverse transcription-deficient and integration competent and the second vector is integration-deficient and reverse transcription-competent.

In this embodiment, the combination of the vectors ensure a homogenous TOI expressing cell population. This embodiment ensures that there are no cells in a transduced cell population which express either the first or second transgene alone. The specific mix-match combinations of a kit of vectors where the first vector comprises a first TOI and the second vector comprises a second TOI are listed in FIG. 6.

Transgene of Interest (TOI)

A transgene of interest (TOI) is a gene or genetic material which has been transferred e.g. by a vector delivery system, such as a retroviral or lentiviral vector delivery system from one organism to another (i.e., a host cell), such that the host cell expresses the particular gene of interest.

At least one vector of the kit of vectors of the present invention comprises at least one TOI. The kit may comprise TOIs on each vector of the kit of vectors. The TOI of the present invention may be any gene that is desired to be expressed in the transduced cell population. The TOI, for example, may comprise a nucleic acid sequence encoding a chimeric antigen receptor (CAR) or an engineered T cell receptor (TCR) and/or a suicide gene (e.g., RQR8). The TOI may also be an enhancer which enhances expression and/or function of the co-expressed TOI, such as increasing the persistence of a CAR. A cell population which does not require sorting or cell selection and comprises increased CAR expression/improved persistence may be advantageous in a clinical setting. An enhancer TOI may be co-expressed with a CAR TOI on the same vector or on a different vector within the kit.

TOI: Chimeric Antigen Receptors

CARs, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A trans-membrane domain anchors the protein in the cell membrane. A CAR may comprise or associate with an intracellular T-cell signalling domain or endodomain.

CAR-encoding nucleic acids may be transferred to cells, such a T cells, using the retroviral or lentiviral vector of the present invention. In this way, a large number of cancer-specific T cells can be generated for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

TOI: Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

The transgenic TCR for use in the present invention may recognise a tumour associated antigen (TAA) when fragments of the antigen are complexed with major histocompatibility complex (MHC) molecules on the surface of another cell.

TOI: Suicide Gene

A suicide gene encodes a polypeptide which enable the cells expressing such a polypeptide to be deleted, for example by triggering apoptosis. An example of a suicide gene is described in WO2013/153391.

TOI: Enhancer

An enhancer encodes any molecule which enhances the expression and/or function/phenotype of another TOI. An enhancer may be co-expressed with the TOI that encodes the molecule upon which it has an effect (e.g. a CAR). The cell may be transduced with the co-expressed TOIs on the same vector or on separate vectors of the kit.

Examples of known enhancers with respect to the CAR may comprise, for example, cytokines and transcription factors. Expression of cytokines (e.g. IL-12) for improved functionality of CAR-T cells has shown the ability to release IL-12 at tumour site, improving anti-tumour activity by shutting down tumour antigen expression (Chmielewski M et al., Cancer Res. 2011; 71:5697-706).

Another example of a CAR enhancer TOI may comprise a transcription factor which generates a specific cell phenotype. For example, the overexpression of T-bet can induce a Th1-like phenotype in CD4+ T cells, conferring higher expression of the CAR, elevated secretion of Th1 and proinflammatory cytokines, and improved cellular cytotoxicity against target-expressing tumour cells (Sentman et al 2018 Cancer Gene Therapy: 25, 17-128).

Another example of a CAR enhancer TOI may comprise a dominant negative SHP2 (dnSHP2) molecule. This truncated peptide has been shown to block the function of endogenous inhibitory phosphatase (PD-1), an inhibitory signal released by the tumour microenvironment and restore function of cytotoxic CAR T cells (Balden V et al., 2017 Blood: 130: 3190).

Another example of a CAR enhancer TOI may encode a secreting factor comprising a mutant or truncated transforming growth factor beta (TGFP) capable of binding a TGFβ Receptor (TGFβR). This factor has been shown to disrupt the interaction of a CAR T cell with TGFβ (as well as other immune effector cells). This disruption permits modulation of the inhibitory microenvironment and therein prevents inhibition of the CAR T cell. This in turn augments the ability of the CAR T cell to attack the tumour.

Importantly, it may be undesirable to express a CAR enhancer (T011) (e.g., dnSHP2 or TGFβ) without a CAR (TOI2). In the first embodiment, provided herein is a method of ensuring the cell population expresses only the combination of CAR and enhancer (TOI1 and TOI2) or CAR (TOI2) on its own (FIG. 6). In contrast with TOI1, expression of CAR on its own may not contribute to a risk of undesirable clinical responses such as cytokine storm. Therefore, there is no need to sort the cells for expression of TOI2 without TOI1.

TOI: Reporter Gene/Detectable Marker

Reporter genes can be fluorescent proteins, such as Green Fluorescent Protein (GFP) or mutants of GFP (e.g. BFP, CFP, YFP). Fluorescent proteins are known to be used as a reliable and quantitative reporter of gene expression when measured by flow cytometry. These genes can be used to exemplify methods, as shown in the examples herein.

Other examples of similar fluorescent proteins include but are not limited to mCherry, mNeptune, mOrange, mWasabi, mTurquoise, TagRFP657, TagBFP. See list http://nic.ucsf.edu/dokuwiki/doku.php?id=fluorescent_proteins.

Integrase (IN)

IN is a protein generated by protease (PR)-mediated cleavage of the C-terminal portion of the retroviral Gag pol polyprotein. IN catalyses reactions by modifying the ends of both the genomic and recombinant DNA, allowing their subsequent ligation, thus incorporating the recombinant DNA into the host genome. IN is required for the generation of functional viruses, capable of successful incorporation of recombinant DNA into target cells.

IN is composed of three domains: N-terminal domain (NTD), catalytic core domain (CCD), and C terminal domain (CTD). The NTD is well conserved amongst retroviruses with respect to a motif (HHCC) making up the zinc-binding site, and is known to play an important role in the key steps of integration (Saeed et al., Mol Ther Nucleic Acids. 2014: 3(12) 213). The CCD is responsible for providing the structural framework for catalysis, and comprises a triad of critically conserved residues. For example, the DDE motif in HIV-1 (Wanisch and Munoz, Mol Ther. 2009; 17(8): 1316-1332).

By deleting or mutating all or part of any one or all of the three domains of the IN, the integration activity of IN of the viral vector can be abolished.

Integration-Deficient Vector and Integration-Competent Vector

An integration-deficient vector is a viral vector which lacks the ability to integrate a transgene of interest (TOI) into the host cell. The ability to integrate the TOI is removed from the viral vector by abolishing the activity of the IN produced by the Gag pol polyprotein. Therefore, the integration-deficient vector lacks a functioning IN, and alone, is not able to integrate the TOI of the vector into the genome of the infected cell.

An integration-competent vector is a viral vector (or virus) which has the ability to integrate a TOI into the host cell.

The integration-competent vector may also be a reverse transcription-deficient vector or reverse transcription-competent vector. Additionally, a reverse transcription-competent vector may also be an integration-deficient vector or an integration-competent vector.

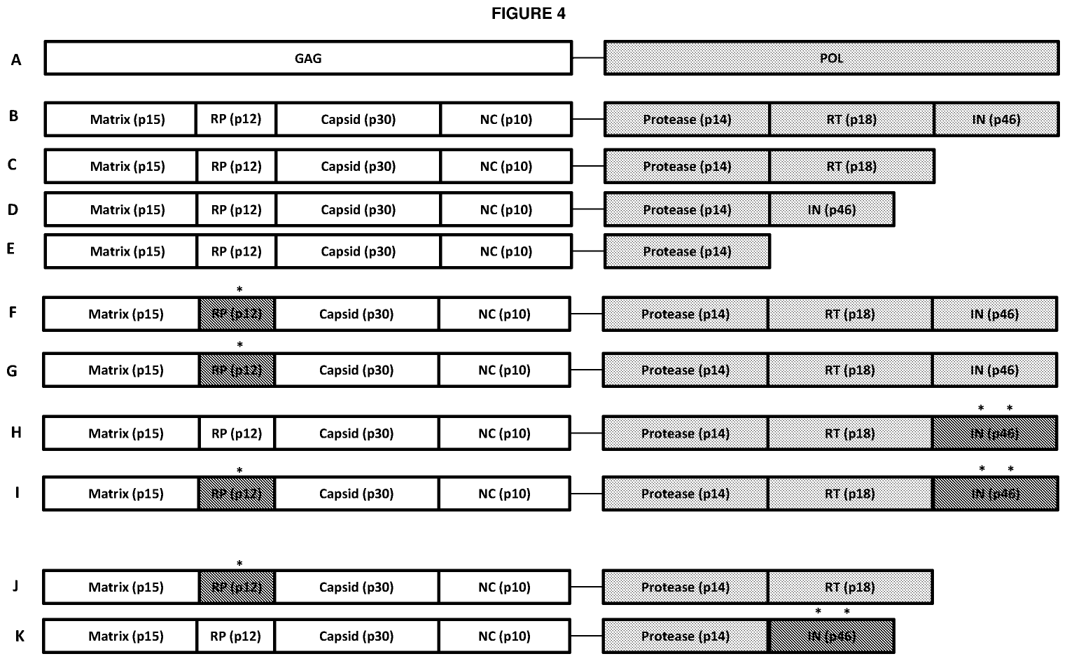

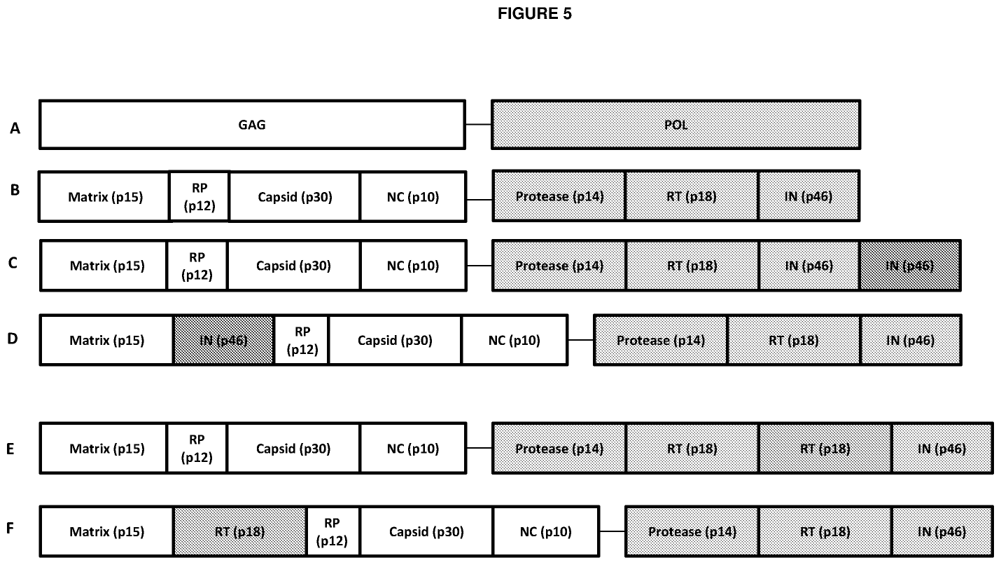

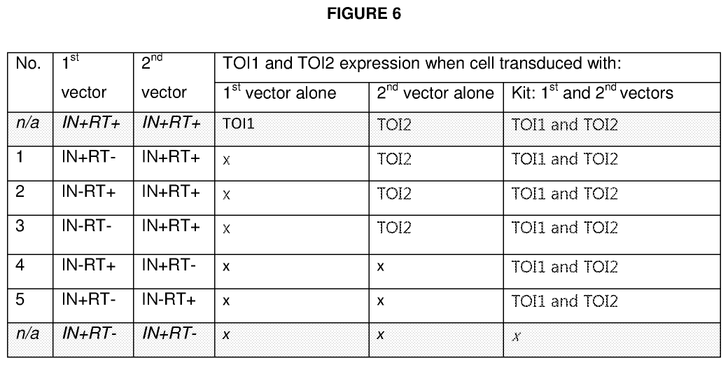

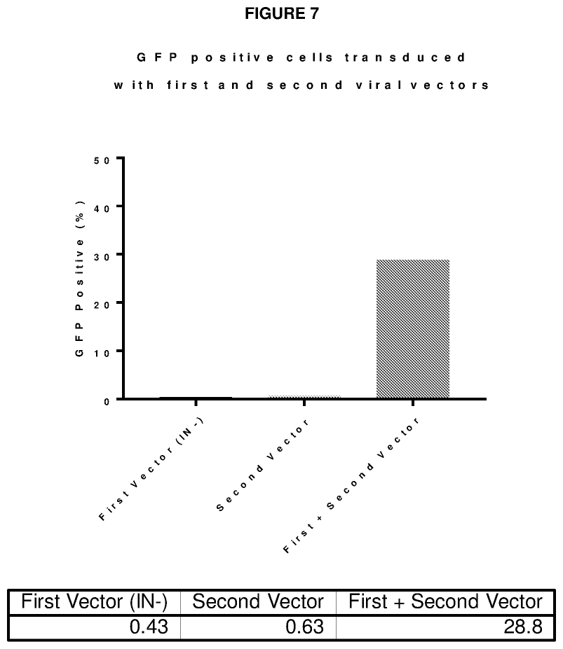

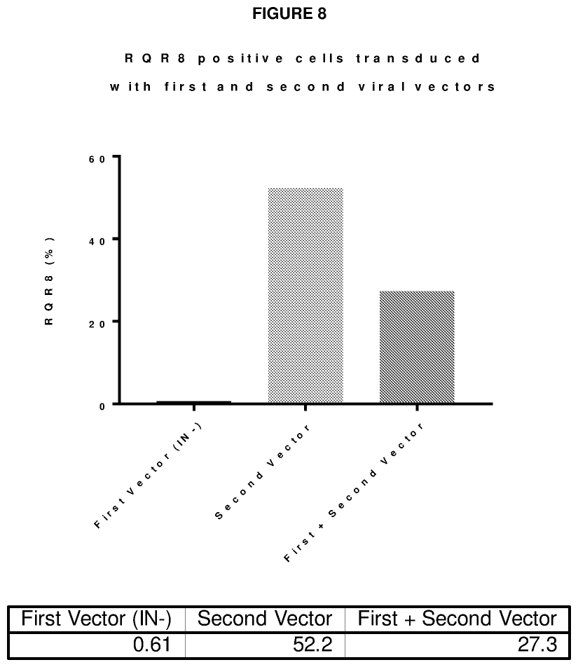

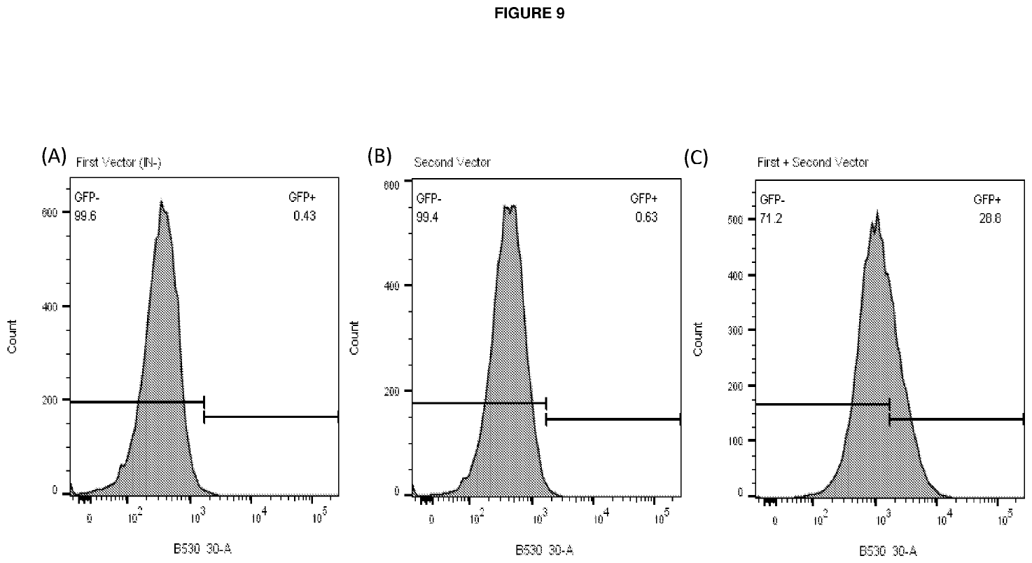

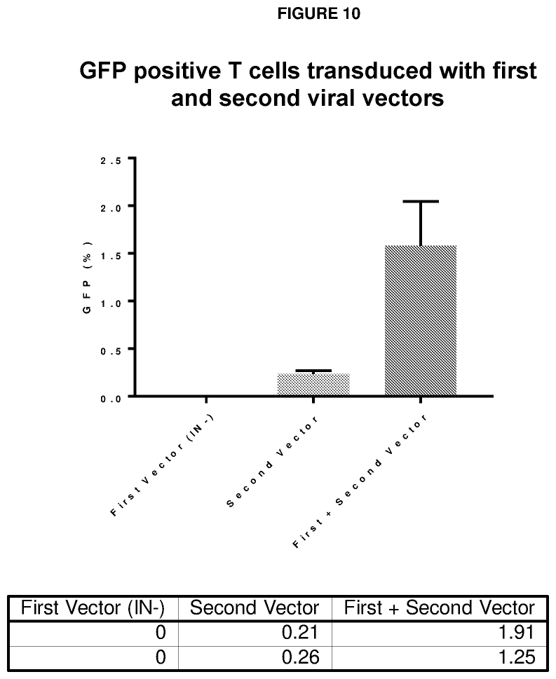

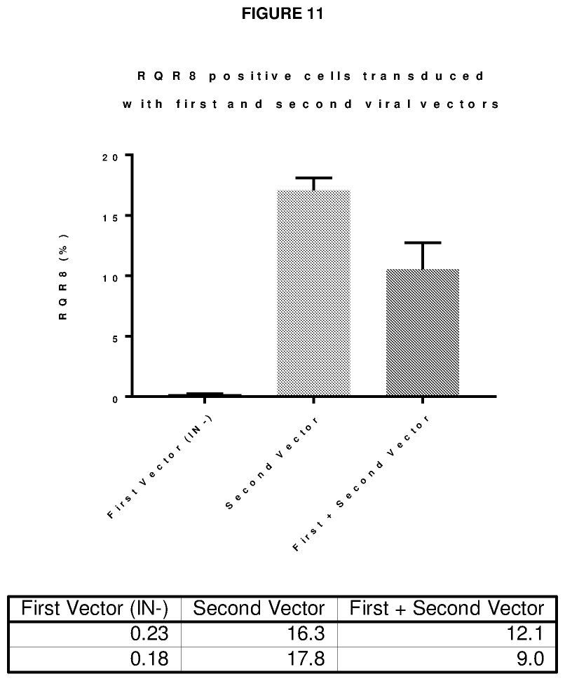

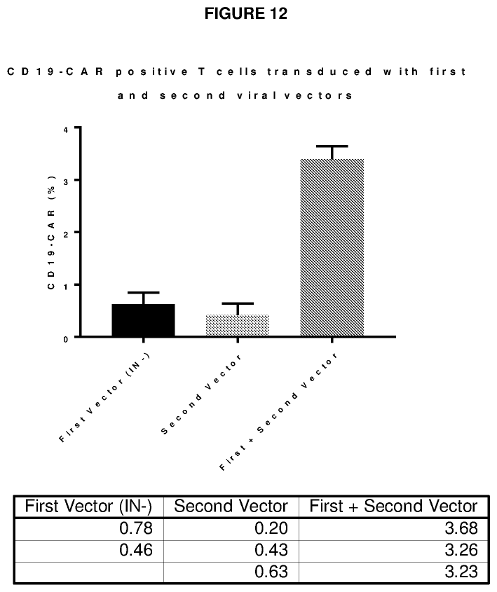

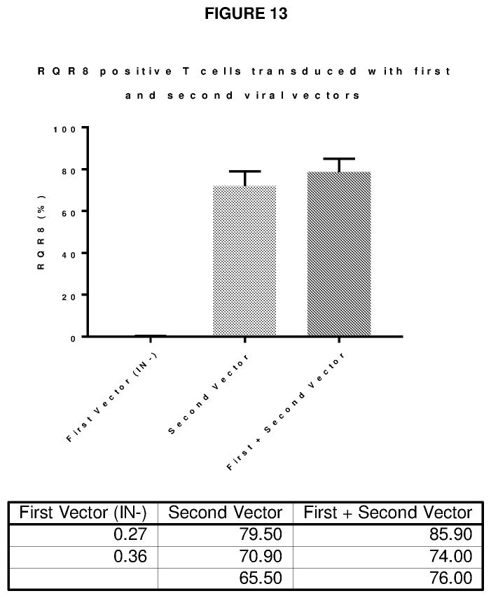

The integration-competent vector may comprise a wild type (WT) Gag pol polyprotein construct, which comprises IN (FIGS. 4A and 4B). In addition, the integration competent vector may also be reverse transcription-deficient (IN+RT−) and therein comprise deleted RT and/or mutated RNA binding phosphate region (FIGS. 4D, 4F, 4G).

The inventors found that the integration activity of the IN in the integration-competent vector restores or “rescues” the integration activity of the IN lacking in the integration-deficient vector. This advantageous feature permits integration of the genome of the integration-deficient vector in the presence of the integration-competent vector.

Additionally or alternatively, the integration-competent vector may comprise a mutated Gag pol polyprotein construct such that additional IN enzyme is produced by the polyprotein. For example, an additional IN-encoding sequence can be provided at the C-terminal end (FIG. 5C) or at the N-terminal end of the Gag pol polyprotein construct (FIG. 5D).

Described herein is a kit of vectors comprising an integration-deficient vector which lacks the ability to integrate a TOI into a host cell and an integration-competent vector which has the ability to integrate a TOI into a host cell. There may be more than one integration-deficient vector in the kit. There may be more than one integration-competent vector in a kit.

Preferably, the first vector is an integration-deficient vector and the second vector is an integration-competent vector.

Removing IN function of a viral vector to prepare the integration-deficient vector of the present invention may be achieved in a number of ways described herein.

Integration-Deficient Vector Lacking In

Described herein is an integration-deficient vector lacking the IN enzyme, wherein the portion of the sequence encoding the Gag pol polyprotein construct which produces the IN is removed, such that the integration activity of the viral vector is abolished. FIGS. 4C, 4E and 4J illustrate a Gag pol polyprotein construct lacking IN.

Integration-Deficient Vector Comprising Truncated In

Alternatively, the integration-deficient vector may comprise a truncated IN, wherein a part of the IN-encoding sequence is deleted such that the integration activity of the IN is abolished. For example, the sequence encoding one or more of the conserved domains of IN described herein (for example, NTD or CCD) may be fully or partially removed.

Alternatively, any part of the IN-encoding sequence may be removed so long as the integration activity of the viral vector comprising the truncated IN is abolished.

Integration-Deficient Vector Comprising Mutated In

One or more amino acid substitutions may be introduced to the IN sequence such that integration activity of the IN is abolished. For example, the IN sequence may comprise amino acid mutations in one or more of the conserved domains described here. Alternatively, amino acid mutations may be introduced anywhere in the viral vector such that the function of the IN is abolished. Table 1 provides some examples of IN amino acid substitutions of the MLV and HIV-1 Gag pol polyprotein constructs.

TABLE 1

| IN MLV (Retrovirus): UniProt | IN HIV-1 (Lentivirus): UniProt |
|---|---|
| P03355^{\|1331-1738\|} | Q76353 |
| D184A, K376A | D116A, D64V, E152 |

Integrase Inhibitor (INI)

Integrase inhibitors (INI) are a class of antiretroviral drug designed to block the action of IN. INIs were initially developed for the treatment of HIV infection. Known examples of INIs are raltegravir, dolutegravir, elvitegravir used in the treatment of HIV (see Yang et al., 2013). There are known INI resistance mutations (Blanco et al., J Infect Dis. 2011; 203(9): 1204-1214 and http://hivdb.stanford.edu/DR/INIResiNote.html).

An integration-deficient vector may beprovided by means of administration of an IN inhibitor (INI), wherein at least one of the other vectors of the kit may be adapted to be resistant to the INI by introducing an INI resistant mutation.

This arrangement provides a kit of vectors comprising an integration-deficient vector and an integration-competent vector, wherein both vectors are subjected to an INI, but IN activity is only inhibited by the INI in the integration-deficient vector.

Reverse Transcriptase (RT)

Reverse transcriptase (RT) is an enzyme used to generate double-stranded (ds)DNA from an RNA template, a process called reverse transcription. RT are used by retroviruses to replicate their genomes.

Retroviral RT has three sequential biochemical activities: RNA-dependent DNA polymerase activity, ribonuclease H, and DNA-dependent DNA polymerase activity. The dsDNA generate by these collective activities then integrates into the host genome, from which new RNA copies can be made via host-cell transcription. Retroviral RTs have a domain belonging to the RNase H family, which is vital to their replication.

Examples of well studied RTs comprise: HIV-1 RT (UniProt: Q9WJQ2) which has two subunits, which have respective molecular weights of 66 and 51 kDa; M-MLV RT (UniProt: Q83371) from the Moloney murine leukemia virus, which is a single 75 kDa monomer; and AMV RT (UniProt: Q83133⁴⁷⁻²³⁵) from the avian myeloblastosis virus, which also has two subunits, a 63 kDa subunit and a 95 kDa subunit.

Reverse-transcribing RNA viruses, such as retroviruses, use the enzyme to reverse-transcribe their RNA genomes into DNA, which is then integrated into the host genome and replicated along with it.

RNA Binding Phosphotase (RP, p12)

RP is involved in both virus assembly and early post entry steps (Yueh and Goff., J Virol. 2003 February; 77(3): 1820-1829). MLV-derived RP (p12) is 84 amino acid residues in length, with a PPPY motif essential for the efficient release of virion particles. The C-terminus of p12 comprises an arginine-rich stretch essential for infection, and deletion of N-terminus portion of p12 result in a block at an early stage of infection. Alanine substitution at both the N and the C termini of p12 causes defects in events such as reverse transcription and DNA integration (Crawford et al., 1984: J. Virol. 49:909-917). In particular, p12 phosphorylation at serine residues is shown to correlate with virion maturation and modulate RNA-binding activity and viral replication.

Phosphorylation of p12 is important in the process of virus infection prior to integration. S61 is required for nearly all p12 phosphorylation.

By deleting and/or mutating any part or all of the features of RT and or RP described above, replication and therefore ultimately the integration of the TOI of the viral vector is abolished, and the vector is reverse-transcription-deficient.

Reverse Transcription-Deficient Vector and Reverse Transcription-Competent Vector

A reverse transcription-deficient vector is a viral vector which lacks the ability to reverse transcribe the viral RNA to generate linear DNA. This results in failure of the TOI to make a copy of itself and therefore the transgene is not able to integrate into the host cell.

The ability to integrate the TOI into the host cell may be removed from the viral vector by abolishing the activity of the RT produced by the Gag pol polyprotein (FIG. 4D). Alternatively or in addition, failure to integrate into the host cell may be achieved by mutating the RNA Binding Phosphotase protein (RP) produced by the Gag protein of the Gag pol polyprotein (FIGS. 4F and 4G).

The reverse transcription-deficient vector lacking a functioning RT and/or comprising a mutated RP is alone, not able to integrate a TOI of the vector into the DNA of an infected cell.

At least one of the vectors of the kit of the present invention may be a reverse transcription-deficient vector and one of the other vectors of the kit may be reverse transcription-competent. The reverse transcription-deficient vector and the reverse transcription-competent vector are not the same vectors.

The reverse transcription-competent vector is a viral vector which may comprise a WT Gag pol polyprotein construct (FIGS. 4A and 4C). The reverse transcription-competent vector comprises RT, and ultimately permits the TOI to integrate into the host cell by providing the means for the TOI to replicate. A reverse transcription-competent vector may be also integration-competent or integration-deficient. In addition, the reverse transcription-competent vector is a viral vector which comprises a non-mutated RP.

Alternatively, the reverse transcription-competent vector is a viral vector which comprises a mutated Gag pol polyprotein construct such that additional RT enzyme is produced by the polyprotein. For example, an additional RT sequence can be provided at the C-terminal end (FIG. 5E) or at the N-terminal end of the Gag pol polyprotein construct (FIG. 5F).

Importantly, the reverse transcription-competent vector has the ability to integrate TOI(s) of the reverse transcription-deficient vector as well as the reverse transcription-competent vector of the kit. As exemplified, host cell expression of a reverse transcription-deficient vector TOI (e.g., GFP) can be visualised by flow cytometry in the presence of a reverse transcription-competent vector.

Described herein is a kit of vectors comprising a reverse transcription-deficient vector (e.g., a first vector) which lacks the ability to integrate a TOI into a host cell and an reverse transcription-competent vector, (e.g., a second vector) which has the ability to integrate a TOI into a host cell. There may be more than one reverse transcription-deficient vector in the kit. There may be more than one reverse transcription-competent vector in a kit. Removing RT function of a viral vector to make the reverse transcription-deficient vector of the present invention may be achieved in a number of ways described herein.

Reverse Transcription-Deficient Vector Lacking RT

Described herein is a reverse transcription-deficient vector lacking the RT enzyme, wherein at least the part of the sequence encoding Gag pol polyprotein construct which produces the RT is removed, such that the reverse transcription activity of the viral vector is abolished. FIGS. 4D, 4E and 4K illustrate a Gag pol polyprotein construct lacking RT.

Reverse Transcription-Deficient Vector Comprising Truncated RT

Alternatively, the reverse transcription-deficient vector may comprise a sequence encoding a truncated RT, wherein at least part of the RT-encoding sequence is deleted such that the integration activity of the RT is abolished. For example, sequences encoding the domains belonging to the RNase H family, which is vital to their replication may be fully or partially removed. Alternatively, any part of the RT-encoding sequence may be removed such that the reverse transcription activity of the viral vector comprising the truncated RT is abolished.

Reverse Transcription-Deficient Vector Comprising Mutated RP

One or more amino acid substitutions may be introduced to the RP sequence produced by the pol protein of the Gag pol polyprotein construct such that replication activity of the reverse transcription is abolished. For example, the RP sequence may comprise amino acid mutations in one or more of the conserved domains and/or motifs described herein.

Table 2 provides some examples of RP (p12) amino acid substitutions that abolish the effect of a functioning RP, thus providing a reverse transcription-deficient vector of the present invention.

TABLE 2

MLV (retrovirus): P03355^{|660-1330|} (p12)
S192A, S192D, S61A

Specific Combinations of Vectors for TOI Expressing Cells

When the vector is both integration competent and reverse transcription competent (IN+RT+) the vector may be wild type Gag pol. This vector is capable of rescuing a second vector of the kit which is deficient in integration and/or reverse transcription activities. Effectively, this first vector (IN+RT+) will make up for any IN or RT deficiencies of the second vector such that the TOI on the first or second vector is expressed in the host cell.

When a vector is integration-deficient and reverse transcription-competent (IN−RT+), the vector may comprise Gag pol having either a deleted or mutated IN. However, the region encoding either RT or RNA binding phosphatase is neither deleted nor mutated and therefore remains functional.

When a vector is integration-deficient and reverse transcription-deficient (IN−RT−), the vector may comprise Gag pol having both deleted/mutated IN and deleted or mutated RT or RNA binding phosphatase. In this arrangement, neither the integration nor the reverse transcription activity of the vector is competent and is not able to rescue a second vector of the kit which is either integration or reverse transcription deficient.

FIG. 6 lists the vector combinations of the kit in which the first vector comprises a first transgene (TOI1) and the second vector comprises a second transgene (TOI2). The vectors may be integration competent (IN+) or integration deficient (IN−) and reverse-transcription competent (RT+) or reverse transcription deficient (RT−) and the effect of single transduction (1^{st} vector alone and 2^{nd} vector alone) and double transduction (1^{st} and 2^{nd} vectors) on transgene expression by cells is shown.

In FIG. 6, row numbers 1,2 and 3 are examples of kits of vectors of the first embodiment of the first aspect of the invention; and row numbers 4 and 5 are examples of kits of the second embodiment of the first aspect of the invention.

Cell

There is provided a cell, (e.g., a host cell or target cell) which is transduced with a kit of vectors of the present invention.

The cell may be a cytolytic immune cell such as a T cell or an NK cell or a hematopoietic stem cell HSC.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4₊CD25₊FoxP3₊ Treg cells) arise in the thymus and have been linked to interactions between developing T cells with both myeloid (CD11c₊) and plasmacytoid (CD123₊) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Transduced cells may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, cells may be derived from ex vivo differentiation of inducible progenitor cells or embryonic progenitor cells to, for example, T or NK cells. Alternatively, an immortalized T-cell line which retains its lytic function and could act as a therapeutic may be used.

In all these options, TOI-expressing cells are generated by introducing DNA or RNA coding for the TOI by one of many means including transduction with a kit of vectors of the invention.

The cell of the invention may be an ex vivo cell from a subject. The cell may be from a peripheral blood mononuclear cell (PBMC) sample. Such cells may be activated and/or expanded prior to being transduced with nucleic acid encoding the molecules providing the kit of vectors according to the first aspect of the invention, for example by treatment with an anti-CD3 monoclonal antibody.

The cell of the invention may be made by:
(i) isolation of a cell-containing sample from a subject or other sources listed above; and
(ii) transducing the cell with a kit of vectors according to the first aspect of the invention.

Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
a) isolation of a cell-containing sample from a subject
b) transducing the cell-containing sample with a kit of vectors according to the first aspect of the invention
c) culturing the cell-containing sample to enable integration of all of the vectors of the kit,
d) administering the TOI expressing cell-containing sample to the subject.

The present invention provides a cell composition for use in treating and/or preventing a disease.

The invention also relates to the use of a pharmaceutical composition comprising a population of transduced cells as described above in the manufacture of a medicament for the treatment and/or prevention of a disease.

The disease to be treated and/or prevented by the methods of the present invention may be a cancerous disease, such as Acute lymphoblastic leukemia (ALL), chronic lymphoblastic leukemia (CLL), bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

The cells and pharmaceutical compositions of present invention may be for use in any of the methods described above.

Making a Retroviral Vector: Transient Transfection

The HEK293 human embryonic kidney-derived cell line may be used as a host cell for vector production as it is well characterised and highly transfectable. A host cell may be any mammalian cell type capable of producing retroviral/lentiviral vector particles. The host cells may be 293T-cells, or variants of 293T-cells which have been adapted to grow in suspension and grow without serum.

To avoid the generation of replication-competent retrovirus (RCR) or lentivirus (RCL), the vector components are split into three or four plasmids: (i) the helper plasmid containing the Gag-Pol coding sequence, (ii) the envelope plasmid containing the Env coding sequence, the (iii) genome plasmid containing the TOI and in the case of lentiviral vector production an additional (iv) rev plasmid containing the Rev accessory sequences (for lentiviral vectors only). The plasmids will be designed to minimise overlapping or homologous DNA regions thus reducing the risk of RCR generation by homologous recombination.

To generate retroviral vectors, the three or four plasmids may be transiently transfected into the host cells. Plasmid entry into the cells may be assisted by the addition of compounds such as calcium phosphate, lipofectamine or polyethylenimine (PEI). The expression of the plasmid components in the transfected host cells results in the generation of recombinant retroviral or lentiviral vectors, which can subsequently be harvested from the supernatant.

The triple (or quadruple) transfection system can be used both for the generation of high titre retroviral (or lentiviral) stock for transduction and for the screening of gag-pol mutants for use in the viral vectors of the present invention (Soneoka et al., Nucleic acid Res, 1995: 23,4: 628-633).

Making a Retroviral Vector: Stable Packaging and Producer Cells

In transient transfection, the plasmids enter the host cell but are not integrated into the cellular genome. This method is generally fast, flexible, and useful for small-scale vector production. In contrast, the generation of stable producer cells where plasmids are integrated into the host cell genome is a longer and more complex process, and useful for large scale vector production.

A packaging cell is a cell that requires transient transfection of only the genome plasmid containing the TOI in order to produce retroviral vectors. The packaging cell for a retroviral vector may comprise integrated copies of gag, pol and env genes. The packaging cell for a lentiviral vector may comprise integrated copies of gag, pol, env and rev genes. The packaging cell may be transiently transfected with the genome plasmid containing the TOI(s), and the recombinant vector can subsequently be harvested from the supernatant.

A producer cell is a cell that contains all of the components required for vector production as genomic integrants. For a retroviral vector, that may comprise gag, pol and env genes and the TOI(s) flanked by the LTRs and regulatory & packaging sequences. For a lentiviral vector, that may comprise gag, pol, env and rev genes and the TOI(s) flanked by the LTRs and regulatory & packaging sequences. The producer cells may be grown in culture and the recombinant vector can be harvested from the supernatant.

Method of Making a Kit of Retroviral Vectors

In the kit of vectors of the present invention, any of the vector production methods described above can be used to make any of the vectors of the kit.

Thus, the method of making a kit of vectors of the present invention comprises the step of generating the first viral vector either by transient transfection or by using stable packaging or producer cells. The viral genome comprises the TOI of the present invention. The TOI may be any one or more of the TOIs described herein. The generation of the first viral vector is completed by culturing the first cell and harvesting the first viral vector from the supernatant.

The second viral vector of the kit of the present invention is generated by transient transfection or by using stable packaging or producer cells. The viral genome of the second viral vector also comprises any one or more of the TOIs described herein. The TOI of the second viral vector may, for example, comprise a nucleic acid sequence encoding for a chimeric antigen receptor (CAR) and a second nucleic acid sequence encoding a suicide gene, such as RQR8. The generation of the second viral vector is completed by culturing the second cell and harvesting the second viral vector from the supernatant.

The invention will now be further described by way of Examples, which are meant to serve to assist one of ordinary skill in the art in carrying out the invention and are not intended in any way to limit the scope of the invention.

Method of Ensuring Cell Population Lacks Single Transgene-Expressing Cells Without Sorting

The present invention also provides a method for making a population of cells comprising:

a sub-population of cells co-express a first transgene and a second transgene; and a subpopulation of cells express the second transgene alone, but lacking a subpopulation of cells expressing the first transgene alone

which comprises the step of transducing cells with a kit of vectors of the first embodiment of the first aspect of the invention.

The present invention also provides a method for avoiding the creation of a subpopulation of cells expressing a first transgene alone when transducing cells with first and second vectors comprising first and second transgenes respectively, which comprises the step of transducing cells with a kit of vectors of the first embodiment of the first aspect of the invention.

The present invention also provides a cell population transduced with a kit of vectors of the first embodiment of the first aspect of the invention which lacks any cells expressing the first transgene alone.

The present invention also provides a method for making a population of cells comprising:

a sub-population of cells co-expressing a first transgene and a second transgene, but lacking a subpopulation of cells expressing the first transgene alone or the second transgene alone

which comprises the step of transducing cells with a kit of vectors of the second embodiment of the first aspect of the invention.

The present invention also provides a method for avoiding the creation of a subpopulation of cells expressing a single transgene alone when transducing cells with first and second vectors comprising first and second transgenes respectively, which comprises the step of transducing cells with a kit of vectors of the second embodiment of the first aspect of the invention.

The present invention also provides a cell population transduced with a kit of vectors of the first embodiment of the second aspect of the invention, which lacks any cells expressing the first or second transgene alone.

### EXAMPLES

### Example 1: Preparation of a Mutant Gag pol Construct: IN Removal

Golden Gate cloning was used to create a mutant Gag pol-encoding sequence unable to produce a functional IN. The two regions flanking the IN-encoding sequence were amplified by PCR using primers containing sequences for a Type IIS restriction endonucleases. The PCR products were then digested with the Type IIS restriction endonucleases to yield compatible sticky ends for subcloning into the original vector. The wild type GagPol plasmid was digested with type II restriction enzymes to yield compatible ends for ligation of the digested PCR products. Following ligation, the new plasmid was transformed into competent bacteria cells and sequenced to verify the correct deletion had occurred i.e., the IN-encoding sequence had been successfully removed.

### Example 2: Preparation of a Mutant Gag pol Construct: IN Amino Acid Substitution

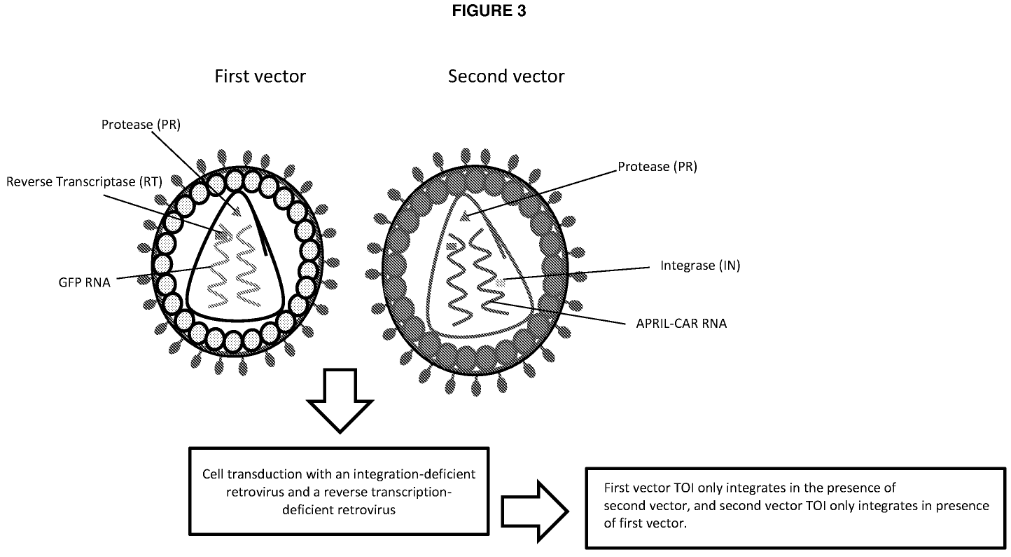

To create a mutant Gag pol sequence which encodes a mutant, non-functional IN including specific amino acid substitutions, a gBlock was ordered containing the IN-encoding sequence producing IN with the required mutations, as described in Table 1 and FIG. 3H. PCR was used to amplify the gBlock, which was then digested with Type II restriction endonucleases for cloning into the original GagPol vector, replacing the existing IN sequence.

### Example 3: Preparation of a Mutant Gag pol Construct: RP Amino Acid Substitution

To mutate the RNA Binding Phosphotase (RP) protein-encoding sequence, site directed mutagenesis was used; the entire plasmid was amplified using primers which contained sequences producing the mutations referred to in Table 2 and/or FIGS. 4F or 4G. The resulting PCR product was treated with a mix of a kinase, DNA ligase and DpnI to circularise the PCR amplified plasmid and remove the remaining original plasmid used for the PCR reaction, and was then transformed into competent bacteria cells. The resulting modified plasmid was sequenced to verify the introduction of the RP mutations.

### Example 4: Expression of an Integration-Deficient Vector GFP TOI on 293T Cells Transduced in the Presence of an Integration-Competent Vector

HEK293T cells (also known as 293T cells) were seeded at a density of 250,000 cells per well of a 24 well plate. The 293T cells were transduced with a first vector alone at a multiplicity of infection (MOI) of 10, where the first vector is an integration-deficient vector comprising a Gag pol construct lacking IN and further comprising a GFP TOI (GFP lacking IN); a second vector alone at a MOI of 0.5, where the second vector is an integration- and reverse transcription-competent vector, comprising a wild type Gag pol construct and a first TOI comprising a nucleic acid sequence encoding an chimeric antigen receptor comprising APRIL and a second TOI comprising a nucleic acid sequence encoding a suicide gene (RQR8) or a combination of both first and second vectors in the presence of 8 μg/ml polybrene.

16 hours later, the media was changed to remove the residual virus. Then 48 hours post transduction, the expression of GFP was assessed by flow cytometry.

FIG. 7 shows that none of the cells transduced with the first vector alone expressed GFP, whereas a significant proportion of cells expressed GFP when the cells were transduced with a combination of both first and second vectors. The bar graphs of FIGS. 7 and 8 similarly show the combination of both viral vectors yields GFP expression or RQR8 expression, respectively. The resulting transduced 293T-cells of FIG. 8 are a mixture of cells transduced with the second vector alone (RQR8 positive) or double transduced with both vectors (positive for GFP). The mixture of cells transduced does not contain the first vector alone since the first vector is not able to integrate the TOI into the cell genome.

The cell population comprising doubly transduced cells are identified by observing the percentage of cells positive for GFP by flow cytometry (FIG. 7).

### Example 5: Expression of an Integration-Deficient Vector GFP TOI on T Cells Transduced in the Presence of an Integration-Competent Vector

Leukapheresis was thawed and cells are cultured in TexMACS media supplemented with human serum overnight.

The following day cells are analysed for expression of CD45 and CD3 by flow cytometry. Following this, cells are activated with TransACT as per manufacturer's instructions. Cell culture media is additionally supplemented with cytokines.

48 hours following activation, cells are transduced with vectors at the required MOIs as follows: Vectors are added to 24 well plates which have been coated with Retronectin as per the manufacturer's instructions. Plates are then centrifuged for 40 minutes at 1,000g. Following centrifugation, cells are added to 24 well plates in the presence of the appropriate vectors and cultured overnight.

The activated T-cells cells were transduced with a first vector alone at a multiplicity of infection (MOI) of 10, where the first vector is an integration-deficient vector comprising a Gag pol construct lacking IN and further comprising a GFP TOI; a second vector alone at a MOI of 0.5, where the second vector is an integration- and reverse transcription-competent vector, comprising a wild type Gag pol construct and a TOI comprising a nucleic acid sequence encoding an chimeric antigen receptor comprising APRIL and a second TOI comprising a nucleic acid sequence encoding a suicide gene (RQR8) or a combination of both first and second vectors.

16 hours later the media was changed to remove the residual virus. Then 5 days post transduction, the expression of GFP was assessed by flow cytometry. The cell population comprising doubly transduced T cells are identified by observing the percentage of cells positive for GFP by flow cytometry (FIG. 10).

### Example 6: Expression of an Integration-Deficient Vector CAR TOI on T Vells Transduced in the Presence of Integration-Competent Vector dnSHP2 TOI

Leukapheresis was thawed and cells are cultured in TexMACS media supplemented with human serum overnight.

The following day cells are analysed for expression of CD45 and CD3 by flow cytometry. Following this, cells are activated with TransACT as per manufacturer's instructions. Cell culture media is additionally supplemented with cytokines.

48 hours following activation, cells are transduced with vectors at the required MOIs as follows: Vectors are added to 24 well plates which have been coated with Retronectin as per the manufacturer's instructions. Plates are then centrifuged for 40 minutes at 1,000 g. Following centrifugation, cells are added to 24 well plates in the presence of the appropriate vectors and cultured overnight.

The activated T-cells cells were transduced with a first vector alone at a multiplicity of infection (MOI) of 10, where the first vector is an integration-deficient vector comprising a Gag pol construct lacking IN and further comprising a CAR TOI; a second vector alone at a MOI of 0.5, where the second vector is an integration- and reverse transcription-competent vector, comprising a wild type Gag pol construct and a comprising a nucleic acid sequence encoding an dnSHP2 TOI or a combination of both first and second vectors.

16 hours later the media was changed to remove the residual virus. Then 5 days post transduction, the expression of CAR was assessed by flow cytometry.

### Example 7: Expression of an Integration-Deficient Vector CD19-CAR TOI on T Cells Transduced in the Presence of an Integration-Competent Vector

Leukapheresis was thawed and cells are cultured in TexMACS media supplemented with human serum overnight.

The following day cells are analysed for expression of CD45 and CD3 by flow cytometry. Following this, cells are activated with TransACT as per manufacturer's instructions. Cell culture media is additionally supplemented with cytokines.

48 hours following activation, cells are transduced with vectors at the required MOIs as follows: Vectors are added to 24 well plates which have been coated with Retronectin as per the manufacturer's instructions. Plates are then centrifuged for 40 minutes at 1,000 g. Following centrifugation, cells are added to 24 well plates in the presence of the appropriate vectors and cultured overnight.

The activated T-cells cells were transduced with a first vector alone at a multiplicity of infection (MOI) of 3, where the first vector is an integration-deficient vector comprising a Gag pol construct lacking IN and further comprising a CD19-CAR TOI; a second vector alone at a MOI of 0.5, where the second vector is an integration- and reverse transcription-competent vector, comprising a wild type Gag pol construct and a TOI comprising a nucleic acid sequence encoding an chimeric antigen receptor comprising APRIL and a second TOI comprising a nucleic acid sequence encoding a suicide gene (RQR8) or a combination of both first and second vectors.

16 hours later the media was changed to remove the residual virus. Then 5 days post transduction, the expression of CD19-CAR was assessed by flow cytometry. The cell population comprising doubly transduced T cells are identified by observing the percentage of cells positive for CD19-CAR by flow cytometry (FIG. 12).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A kit of vectors comprising a first and second viral vector,
wherein the first viral vector comprises a transgene of interest (TOI), and
presence of the second viral vector in a host cell is required for integration of the first viral vector TOI into the host cell genome.

2. A kit of vectors according to claim 1, wherein the first viral vector is integration-deficient.

3. A kit of vectors according to claim 2, wherein the first viral vector lacks an integrase (IN).

4. A kit of vectors according to claim 2, wherein the first viral vector comprises a truncated IN.

5. A kit of vectors according to claim 2, wherein the first viral vector comprises an IN with one or more amino acid substitution(s).

6. A kit of vectors according to claim 5, wherein the IN is derived from Moloney murine leukemia virus (MLV) and the amino acid substitutions is/are selected from D184N and/or K376A.

7. A kit of vectors according to claim 5, wherein the IN is derived from Human Immunodeficiency virus (HIV) and the amino acid substitution is D116A.

8. A kit of vectors according to according to claim 1, wherein the first viral vector is reverse transcription-deficient.

9. A kit of vectors according to claim 8, wherein the first viral vector lacks a reverse transcriptase (RT).

10. A kit of vectors according to claim 8, wherein the first viral vector comprises a truncated RT.

11. The kit of vectors according to claim 8, wherein the first viral vector comprises a RNA Binding Phosphoprotein (RBP) with one or more amino acid substitutions which abolish(es) RT activity.

12. The kit of vectors according to claim 11, wherein RNA-binding phosphoprotein is derived from MLV, and the amino acid substitution is selected from S192D or S192A.

13. The kit of vectors according to claim 2, wherein the second vector is reverse transcription-deficient.

14. The kit of vectors according to claim 8, wherein the second vector is integration-deficient.

15. A kit of vectors according to claim 1, wherein the TOI comprises a nucleic acid sequence encoding a chimeric antigen receptor (CAR) and/or an expression enhancing molecule.

16. A kit of vectors according to claim 1, comprising a first, second and third viral vector.

17. A cell transduced with a kit of vectors according to claim 1.

18. A cell according to claims 17, which is an immune cell.

19. A cell according to claim 18, which is a T cell, natural killer (NK) cell or a hematopoietic stem cell (HSC).

20. A method for making a cell, which comprises the step of transducing a cell with a kit of vectors according to any of claim 1.

21. A method for preparing a composition of cells which comprises the following steps:
a) transducing a cell-containing sample with a kit of vectors according to claim 1;
b) culturing the cell containing sample enabling integration of the vectors
c) preparing a composition of cells which express TOI.

22. A pharmaceutical composition comprising a plurality of cells according to claim 17.

23. (canceled)

24. A method for treating and/or preventing a disease, which comprises the step of administering a composition of cells according to claim 21 to a subject.

25. A method according to claim 24, which comprises the following steps:
a) isolation of a cell-containing sample from a subject,
b) transducing the cell-containing sample with a kit of vectors according to claim 1,
c) culturing the cell-containing sample enabling integration of all of the vectors of the kit, and
d) administering the TOI expressing cell-containing sample to the subject.

26-27. (canceled)

28. A method of making a kit of vectors according to claim 1, comprising generating the first viral vector by using a sequence encoding an integration-deficient and/or reverse transcription-deficient Gagpol.

29. A method for making a kit of vectors according to claim 13, comprising the following steps:
a) generating the first viral vector using a sequence encoding an integration-deficient Gagpol; and
b) generating the second vector using a sequence encoding a reverse transcription-deficient Gagpol.

30. A method for making a kit of vectors according to claim 14, comprising the following steps:
a) generating the first viral vector using a sequence encoding a reverse transcription-deficient Gagpol; and
b) generating the second vector using a sequence encoding an integration-deficient Gagpol.
